(19) United States
(12) Patent Application Publication
(10) Pub. No.: US 2026/0124429 A1
(43) Pub. Date: May 7, 2026

(54) MULTI-LUMEN CATHETER CONTAINING INTEGRAL STRUT AND METHOD OF ITS MANUFACTURE

(71) Applicant: St. Jude Medical, Cardiology Division, Inc., St. Paul, MN (US)

(72) Inventors: Xiaoping Guo, Eden Prairie, MN (US); Xuan Khieu, Maple Grove, MN (US)

(21) Appl. No.: 18/708,138

(22) PCT Filed: Oct. 27, 2022
(86) PCT No.: PCT/US2022/048065

§ 371 Date: May 7, 2024

Related U.S. Application Data
(60) Provisional application No. 63/277,876, filed on Nov. 10, 2021.

(51) Int. Cl. A61M 25/00; A61B 5/00; A61M 25/01

(52) U.S. Cl. CPC A61M 25/005; A61M 25/0012; A61M 25/0045; A61M 25/0147; A61B 5/6856

(57) ABSTRACT

A multi-lumen catheter shaft (55) includes a single piece strut (110) having a central portion defining a central lumen and a pair of opposing radial arms (116a), each defining a respective biased lumen (116). A first tubular layer (113) surrounds the strut, thus defining two opposing side lumens between the first tubular layer and the strut. The strut and first tubular layer may be separately or integrally formed. A second tubular layer (130) may be disposed around the first tubular layer, with an optional reinforcing layer (120) (e.g., a braided metallic mesh layer) disposed between the first and second tubular layers. Radial walls extending from the central portion of the strut may further subdivide the side lumens. Pull wires (10), activation wires, electrical conductors, and the like may be routed through the central, biased, and side lumens.

### CROSS-REFERENCE TO RELATED APPLICATIONS

This application claims the benefit of U.S. provisional application No. 63/277,876, filed 10 Nov. 2021, which is hereby incorporated by reference as though fully set forth herein.

### BACKGROUND

The present disclosure relates generally to catheters that are used in the human body. In particular, the present disclosure relates to an integral strut for use in multi-lumen catheters, including steerable catheters.

Catheters are used for an ever-growing number of procedures. For example, catheters are used for diagnostic, therapeutic, and ablative procedures, to name just a few examples. Typically, the catheter is manipulated through the patient's vasculature and to the intended site, for example, a site within the patient's heart. The catheter carries one or more electrodes for conducting cardiac ablation, diagnosis, or the like, and may also contain additional positioning sensors usable to determine the position and/or orientation of the catheter within the patient's body.

Because the path through the patient's vasculature to the intended site is often long and tortuous, steering forces typically must be transmitted over relatively great distances.

Accordingly, it is desirable for a catheter to have sufficient axial (e.g., column) strength to be pushed through the patient's vasculature via a force applied at its proximal end (“pushability”). It is also desirable for a catheter to transmit a torque applied at the proximal end to the distal end (“torqueability”). Pushability and torqueability (collectively, “maneuverability”) permit a physician to manipulate a catheter to an intended site and then properly orient the catheter. It is also desirable for a catheter to have sufficient flexibility to substantially conform to the patient's vasculature and yet resist kinking as it does so. Kinking is often the result of a localized failure of the material of the catheter when localized stresses exceed the yield strength of the material.

To provide pushability, torqueability, flexibility, and kink resistance, many extant catheters are made of engineering polymer materials reinforced with metallic wire braiding plaits. The characteristics of pushability, torqueability, flexibility, and kink resistance are often in tension with one another, however, with improvements in one requiring compromises in others.

Steerable and variable-radius loop catheters are also known. One example of such a catheter is the Advisor™ VL Circular Mapping Catheter, Sensor Enabled™ (Abbott Laboratories, Abbott Park, IL).

### BRIEF SUMMARY

Disclosed herein is a catheter shaft, including: a single piece strut having a central portion defining a central lumen extending therethrough and a pair of opposing arms extending radially away from the central portion, each of the pair of opposing arms defining a respective biased lumen extending therethrough; and a first tubular layer surrounding the strut and defining two opposing side lumens extending through the catheter shaft between the first tubular layer and the strut.

In embodiments of the disclosure, the first tubular layer is integrally formed with the strut from a single polymeric material.

In alternative embodiments of the disclosure, the strut is made of a first polymeric material, the first tubular layer is made of a second polymeric material different from the first polymeric material, and the strut is bonded to the first tubular layer.

A second tubular layer may surround the first tubular layer. It is contemplated that the critical solid-state thermal transition temperature of the material used to form the second tubular layer will be lower than the critical solid-state thermal transition temperature of the material used to form the first tubular layer and/or the strut.

Optionally, a reinforcing layer, such as a braided metallic mesh, may be disposed between the first tubular layer and the second tubular layer.

A line extending through the biased lumens and the central lumen defines a deflection axis, while a line extending through a center of the central lumen and perpendicular to the deflection axis defines a neutral axis. It is desirable for an area moment of inertia of the strut about the neutral axis to be less than or equal to an area moment of inertia of the strut about the deflection axis. In this regard, a pair of biasing ribbons, such as metallic ribbons, may be positioned along the neutral axis equidistant from the deflection axis.

The strut may also include a pair of walls extending radially away from the central portion to the first tubular layer, thus dividing each of the two opposing side lumens into a respective pair of sub-lumens.

It is also contemplated for the catheter shaft to include a first pull wire extending through a first biased lumen; a second pull wire extending through a second biased lumen; and a loop-activating wire extending through the central lumen. In still further embodiments of the disclosure, a plurality of electrical conductors may extend through at least one of the two opposing side lumens.

Also disclosed herein is a method of manufacturing a catheter shaft, including: forming a single-piece strut having a central portion defining a central lumen extending therethrough and a pair of opposing arms extending radially away from the central portion, each of the pair of opposing arms defining a respective biased lumen extending therethrough; and forming a first tubular layer around the strut to define two opposing side lumens extending through the catheter shaft between the first tubular layer and the strut.

The single-piece strut and the first tubular layer around the strut may be integrally formed from a single polymeric material. Alternatively, the single piece strut may be formed from a first polymeric material, the first tubular layer may be formed from a second polymeric material different from the first polymeric material, and the first tubular layer may be bonded to the single-piece strut.

Embodiments of the disclosure further include forming a second tubular layer around the first tubular layer. It is contemplated that the critical solid-state thermal transition temperature of the material used to form the second tubular layer will be lower than the critical solid-state thermal transition temperature of the material used to form the first tubular layer and/or the strut.

Still further embodiments of the disclosure include forming a reinforcing layer between the first tubular layer and the second tubular layer.

The single-piece strut defines a deflection axis extending through the biased lumens and the central lumen and a neutral axis perpendicular to the deflection axis. The cross-section of the single-piece strut is such that an area moment of inertia of the strut about the neutral axis is less than or equal to an area moment of inertia of the strut about the deflection axis.

The instant disclosure also provides a catheter shaft formed according to a process including: forming a single-piece strut having a central portion defining a central lumen extending therethrough and a pair of opposing arms extending radially away from the central portion, each of the pair of opposing arms defining a respective biased lumen extending therethrough; forming a first tubular layer around the strut to define two opposing side lumens extending through the catheter shaft between the first tubular layer and the strut; disposing a first pull wire in a first biased lumen; disposing a second pull wire in a second biased lumen; and disposing a loop-activating wire in the central lumen.

The single-piece strut defines a deflection axis extending through the biased lumens and the central lumen and a neutral axis perpendicular to the deflection axis. It is desirable for the cross-section of the single-piece strut to be such that an area moment of inertia of the strut about the neutral axis is less than or equal to an area moment of inertia of the strut about the deflection axis.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 illustrates a representative steerable and variable-radius, high-density, circular mapping catheter according to aspects of the instant disclosure.

FIG. 2 is a cross-sectional view taken along line 2-2 in FIG. 1.

FIG. 3A is a perspective view of a portion of a catheter shaft according to aspects of the instant disclosure, with portions removed for illustrative purposes.

FIG. 3B is a side view of the catheter shaft of FIG. 3A, again with portions removed for illustrative purposes.

FIG. 4 is a cross-sectional view taken along line 4-4 in FIG. 3B.

FIGS. 5, 6, and 7 depict cross-sections of a catheter shaft according to various aspects of the instant disclosure.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

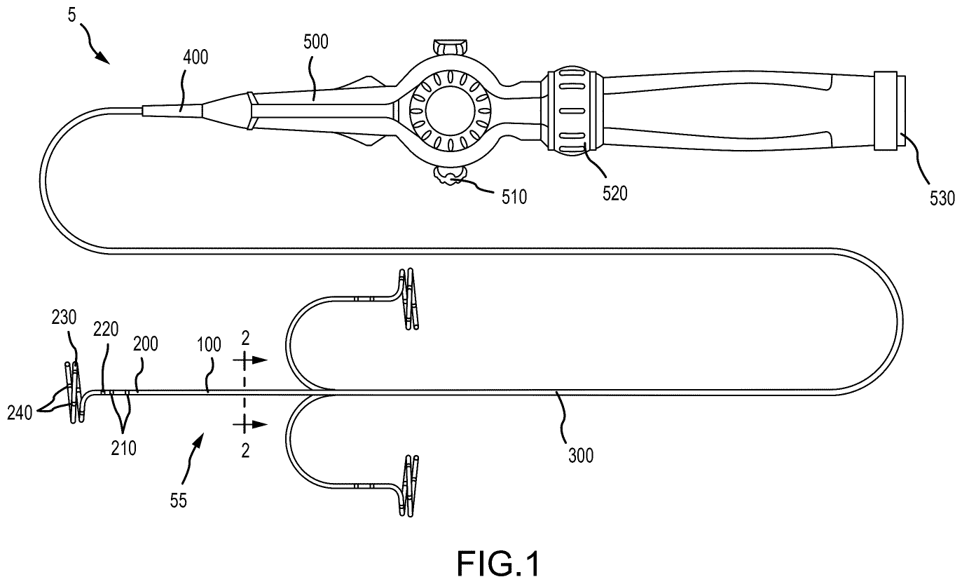

FIG. 1 depicts a representative cardiovascular device, and in particular a catheter 5, such as the Advisor™ VL Circular Mapping Catheter, Sensor Enabled™. It should be understood, however, that the teachings herein can be applied to good advantage in various devices intended to navigate through a patient's vasculature, including, but not limited to, high-density grid electrophysiology mapping catheters (e.g., the Advisor™ HD Grid, Sensor-Enabled™ Mapping Catheter System (Abbott Laboratories, Abbott Park, IL)), irrigated radiofrequency ablation catheters (e.g., the Tacticath™ Sensor Enabled™ Contact Force Ablation Catheter system (Abbott Laboratories, Abbott Park, IL)), and catheters for irreversible electroporation and cryoablation therapies. Insofar as numerous aspects of the construction of catheter 5 will be familiar to those of ordinary skill in the art, however, catheter 5 will only be described in detail herein to the extent necessary to understand the instant disclosure.

In general, catheter 5 includes a distal functional segment (e.g., circular loop segment 230 in FIG. 1) attached and electromechanically coupled to an elongate shaft 55, which in turn includes several interconnected shaft segments (mentioned here in order of the most distal segment to the most proximal segment): a distal shaft segment 200, a deflectable shaft segment 100, and a proximal shaft segment 300. Proximal shaft segment 300 is coupled to a handle 500.

To functionally facilitate high-density circular mapping (e.g., of cardiac activity), a plurality of electrodes (e.g., ring electrodes 240) are disposed along circular loop segment 230. An additional plurality of electrodes (e.g., ring electrodes 210) may be disposed along distal shaft segment 200. Distal shaft segment 200 can further include a magnetic positioning sensor 220 to localize (that is, to determine the position and/or orientation of) catheter shaft 55, for example, to facilitate electroanatomical mapping using an electroanatomical mapping system such as the EnSite Precision™M Cardiac Mapping System (Abbott Laboratories, Abbott Park, IL).

Ring electrodes 210, 230 and magnetic positioning sensor 220 are coupled (e.g., via laser welding) to corresponding separate conductors (e.g., electrical wires). These conductors are systematically laid out within and routed through catheter shaft 55 (e.g., from circular loop segment 230 or deflectable shaft segment 100 through proximal shaft segment 300), until their termination within handle 500, where they may be grouped and coupled to an electrical adapter 510 for interconnection with an electroanatomic mapping system.

Circular loop segment 230 can be formed into a desired preset loop shape through the use of a shaping ribbon (not shown in FIG. 1) made of a superelastic metallic alloy material such as Nitinol. This shaping ribbon is disposed within, and coupled distally with, circular loop segment 230.

As those of ordinary skill in the art will readily appreciate, the loop shape of circular loop segment 230 can be rendered variable through the use of a loop-activating wire 20, the distal portion of which is welded or affixed to the shaping ribbon within circular loop segment 230 and the proximal portion of which is routed through catheter shaft 55 with its proximal end coupled to a loop actuator 520 within handle 500. Thus, manipulation of loop actuator 520 can pull or compress loop-activating wire 20 to uncoil or tighten the distally coupled shaping ribbon and circular loop segment 230 together. This will increase or decrease the radius of curvature of circular loop segment 230, thus allowing circular loop segment 230 to conform to the interior walls of variously sized anatomical features to facilitate circular electrophysiological mapping. At least a portion of loop-activating wire 20 may be enclosed within a compression coil 30 to prevent loop-activating wire 20 from buckling within catheter shaft 55 under compressive loads.

Catheter 5 further includes a deflectable shaft segment 100 and a deflection mechanism operable to deflect deflectable shaft segment 100 from its neutral configuration, shown in solid lines in FIG. 1, into a deflected configuration, shown in phantom in FIG. 1. Such deflectability imparts distal steerability to facilitate the navigation of catheter shaft 55 through the tortuous pathways of a patient's vasculature to a desired location within the patient's heart.

Those of ordinary skill in the art will recognize that a typical deflection mechanism for catheter 5 includes a pair of diametrically-opposed pull wires 10 with their distal ends secured (e.g., via welding) to a pull ring (not shown in FIG. 1) embedded within and disposed distally within deflectable shaft segment 100. Pull wires 10 run from the pull ring through deflectable shaft segment 100 and proximal shaft segment 300, and terminate within handle 500, where they are coupled to a deflecting actuator 510. Thus, when deflecting actuator 510 is moved from its neutral position, one pull wire 10 is placed in tension while the other pull wire 10 is placed in relaxation. This, in turn, applies a bending moment to the pull ring, under which axial bending or shaft deflection of deflectable shaft segment 100 within a deflection plane, as will be discussed in greater detail below, takes place.

Proximal shaft segment 300 imparts axial column strength or pushability, torqueability, kink resistance, and the like to catheter shaft 55, and its proximal end is directly coupled with handle 500. A strain relief 400 can be applied over proximal shaft segment 300 at its junction with handle 500 to absorb any stresses and tensions that may arise from operations of catheter 5.

As discussed in further detail below, deflectable shaft segment 100 and proximal shaft segment 300 can include several inner lumens. Various conductors 40 coupled to ring electrodes 210, 240, loop-activating wire 20, pull wires 10, and the like may be systematically disposed and routed through these lumens.

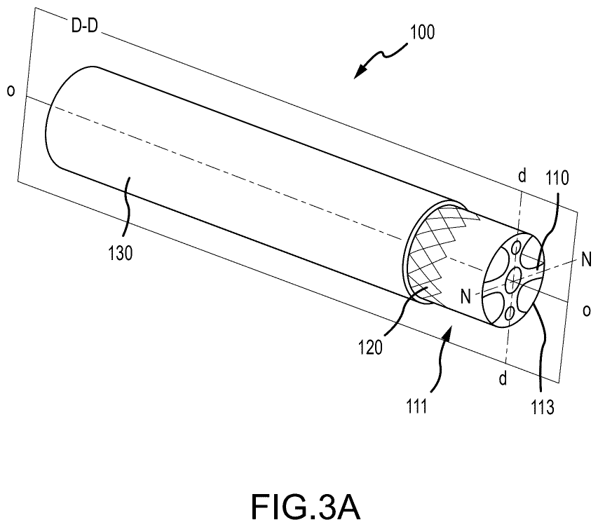

Further aspects of the construction of catheter shaft 55, and in particular of deflectable shaft segment 100, will now be described with reference to FIGS. 3A-6. It should be understood, however, that some or all of the following teachings can also be applied in the construction of proximal shaft segment 300, distal shaft segment 200, and/or circular loop segment 230.

FIG. 3A is a perspective view of deflectable shaft segment 100, while FIG. 3B illustrates a side view of deflectable shaft segment 100; in both cases, portions of deflectable shaft segment 100 have been removed in order to illustrate more clearly the underlying construction thereof. As shown in FIGS. 3A and 3B, deflectable shaft segment 100 includes a strut assembly 111, including a single-piece strut 110 and a first tubular layer 113 surrounding strut 110.

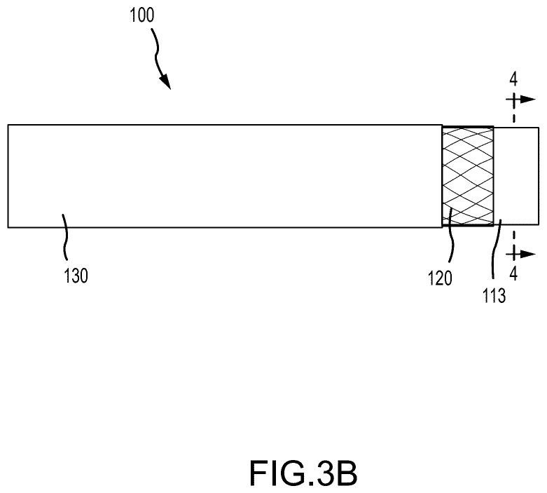

FIG. 4 is a cross-section taken along line 4-4 in FIG. 3B illustrating details of strut assembly 111 according to a first embodiment of the instant disclosure. Strut 110 includes a central portion 114a that defines a central lumen 114 that extends along a longitudinal axis o-o (see FIG. 3A) of deflectable shaft segment 100. Strut 110 further includes a pair of arms 116a extending radially away from central portion 114a, wherein each arm 116a, along with first tubular layer 113, defines a respective biased lumen 116 that also extends along longitudinal axis o-o of deflectable shaft segment 100.

In the embodiment illustrated in FIG. 4, arms 116a are diametrically opposed to each other, thus defining a vertical axis of symmetry, namely deflection axis d-d, within the cross-section of deflectable shaft segment 100. A neutral axis N-N lies perpendicular to deflection axis d-d, through the center of central lumen 114, likewise within the cross-section of deflectable shaft segment 100.

Deflection axis d-d and longitudinal axis o-o of deflectable shaft segment 100 together define a deflection plane D-D (see FIG. 3A), within which deflectable shaft segment 100 axially deflects about neutral axis N-N upon actuation of deflection actuator 510. Deflection of deflectable shaft segment 100 about any axis other than neutral axis N-N, on the other hand, is generally not desirable.

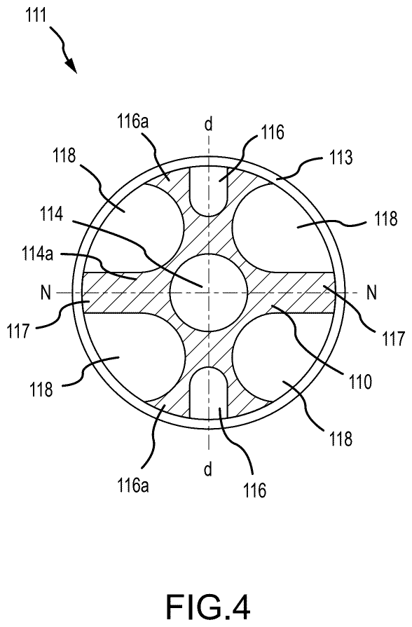

The bending stiffness of strut 110 (and, in turn, of strut assembly 111) about a given axis is a function of the area moment of inertia/about that axis. Accordingly, to facilitate axial deflection about neutral axis N-N within a deflection plane D-D (or along deflection axis d-d) while minimizing any deviation or deflection about an axis other than neutral axis N-N, strut 110 is configured such that its area moment of inertia about neutral axis N-N (I_{N-N}) is less than or equal to its area moment of inertia about deflection axis d-d (I_{d-d}). This, in turn, minimizes or eliminates the need to incorporate additional biasing structures into integral strut assembly 111 (though, as discussed below, such biasing structures, such as stiffeners 115 discussed in connection with FIG. 5, are utilized in certain embodiments of the disclosure).

First tubular layer 113 surrounds strut 110. According to the embodiment depicted in FIG. 4, strut 110 and first tubular layer 113 are respectively and separately formed from a first polymeric material and a second polymeric material different from the first polymeric material, and then integrated with each other to form strut assembly 111 by various manufacturing that will be familiar to those of ordinary skill in the art (e.g., via friction fit, through mechanical interlocking, through the use of adhesives or other chemical bonding, via the application of heat to create a thermal fusion bond or thermal lamination, and the like).

Because first tubular layer 113 surrounds strut 110, a plurality of side lumens 118 are defined between first tubular layer 113 and strut 110. In embodiments of the disclosure, such as the embodiment shown in FIG. 4, strut 110 can also include walls 117 extending radially away from central portion 114a, thereby creating additional side lumens 118 (e.g., the use of two walls 117 results in a total of four side lumens 118 between first tubular layer 113 and strut 110). Those of skill in the art will recognize that, if walls 117 were omitted from the embodiment depicted in FIG. 4, there would be, as illustrated in FIG. 5, two side lumens 118, one on each side of strut 110.

As mentioned above, the use of additional biasing structures, such as biasing stiffeners 115, in order to promote deflection along deflection axis d-d (or within deflection plane D-D) and prevent out-of-plane deflection about an axis other than neutral axis N-N, is contemplated. FIG. 5 is a cross-section of catheter shaft 55 within deflectable shaft segment 100 that depicts a pair of biasing stiffeners 115 positioned along neutral axis N-N within first tubular layer 113. Biasing stiffeners 115 can advantageously take the form of a ribbon of a relatively rigid (e.g., metallic) material having a high Young's modulus extending along longitudinal axis o-o of deflectable shaft segment 100. Biasing stiffeners 115 may have any cross-sectional shape desired, such as flat (as shown in FIG. 5) or round. Insofar as various configurations for biasing stiffeners 115 will be familiar to those of ordinary skill in the art, a detailed description of biasing stiffeners 115 (or analogous stiffening elements) to promote planar deflectability of deflectable shaft segment 100 about neutral axis N-N within deflecting plane (or sweeping plane) D-D is not necessary to an understanding of the instant disclosure. Nonetheless, for purposes of illustration, several exemplary configurations for biasing stiffeners (or analogous stiffening elements) 115 are disclosed in U.S. Pat. No. 7,985,215 , which is hereby incorporated by reference as though fully set forth herein.

As shown in FIG. 4, walls 117 are diametrically opposed to each other and perpendicular to arms 116a, thus creating a horizontal axis of symmetry coincident with neutral axis N-N. Although this configuration is desirable in terms of achieving the relationship between area moments of inertia about deflection axis d-d and neutral axis N-N as described above, and in particular in terms of minimizing or eliminating the need for additional biasing structures, it should be understood that this configuration is merely illustrative and that other configurations may be utilized without departing from the spirit and scope of the instant disclosure (including, as discussed in further detail below, configurations that do utilize additional biasing structures).

Referring again to FIGS. 3A and 3B, a second tubular layer 130 may be disposed over first tubular layer 113 to form deflectable shaft segment 100. Second tubular layer 130 will typically be considerably thicker than first tubular layer 113 that surrounds strut 110. Second tubular layer 130 may also include multiple interconnected or inter-bonded, elastomeric, tubular pieces in different lengths, arranged end-to-end along the length of deflectable shaft segment 100.

FIGS. 3A and 3B also depict a reinforcing braid layer (or metallic mesh) 120 that may optionally be provided between first tubular layer 113 and second tubular layer 130. Reinforcing braid layer 120 may be woven of multiple threads of thin metallic wires in a desired plaiting pattern (e.g., one wire under-over two, two wires over two, over wire over one, etc.), and it may be disposed along some or all of the length of deflectable shaft segment 100.

It is desirable for the material used to form strut 110 to possess a relatively low flexural modulus, a high kink resistance, and a high degree of elastic resilience. It is also desirable that the first and second polymer materials used to form strut 110 and first tubular layer 113 be able to retain their respective, solid-state thermophysical or geometric integrity during structural integration with second tubular layer 130 (and reinforcing braid layer 120, where present) when forming deflectable shaft segment 100 using manufacturing techniques known to those of the ordinary skill in the art. These techniques include polymer reflow, thermal fusion bonding, or thermal lamination.

Thermophysical integrity for a polymer component (e.g., strut 110, first tubular layer 113, and second tubular layer 130) is closely dependent on the critical solid-state thermal transition temperature for its constituent polymer material. Near this temperature, the material will exhibit a critical phase transition, or thermophysical transformation, from a solid or solid-like state to a molten or liquid-like, highly deformative state. With respect to differences in a material's solid-state aggregate structure and relevant thermal transition characteristics, industrial polymer materials include thermoplastic semicrystalline polymers, thermoplastic amorphous polymers, and thermosetting polymers.

Semicrystalline polymers possess a characteristic melting temperature, near which the material will undertake a critical phase transition, or thermal transition, from a solid, semicrystalline state to a molten or liquid state. Amorphous polymers possess a characteristic glass transition temperature, near which the material will experience a critical thermophysical transformation from a solid-like, glassy state to a liquid-like, rubbery state exhibiting high deformability under minimal load.

In contrast, because of its crosslinked polymer structure, thermosetting polymer materials, unlike thermoplastic polymer materials, essentially do not experience a critical, solid-state thermal transition or thermophysical transformation. Instead, their thermophysical integrity of material may only start to gradually deteriorate because of material degradation at relatively high temperatures (e.g., greater than about 300° C. for most thermosetting rubber materials).

Suitable materials for second tubular layer 130 include a variety of melt-processable, elastomer-like polymer materials, namely thermoplastic elastomers (TPEs), which have varying elastic moduli, flexural moduli, and/or durometers to achieve a particular deflectability of deflectable shaft segment 100. It is desirable that selected TPE materials also possess good melt-adhesion to reinforcing braid layer 120 and to the materials used in first tubular layer 113.

For example, TPE materials used to form second tubular layer 130 can be selected from a single or chemically-similar family of block copolymers, such as polyamide 12-based TPE resin materials, namely poly(ether-block-amide) copolymer (PEBA) resins (e.g., Pebax® and Vestamid® E); polyester-based thermoplastic elastomers, namely poly(ether-co-ester) copolymer (PEE) resins (e.g., Hytrel® and Arnitel®); polyether-based thermoplastic polyurethane (TPU) resins (e.g., Pellethane®), Elasthane®, Elastollan®, etc.); polycarbonate-based TPU resins (e.g., Carbothane®, Bionate®, etc.); polydimethylsiloxane-and polyether-based TPU resins (e.g., Pursil®, Elaston-E®, etc.); polydimethylsiloxane-and polycarbonate-based TPU resins (e.g., Carbosil®, etc.), and any combinations thereof.

Styrenic block copolymers, thermoplastic olefin elastomers, and thermoplastic vulcanizates (TPVs), which may be functionalized or grafted with polar chemical groups (e.g., maleic anhydrides, epoxy, acrylic acid, methacrylate, methyl methacrylate, etc.), may be also utilized in second tubular layer 130.

More generally, the materials used to form second tubular layer 130, when properly selected from the above materials and applicable combinations thereof, can have relatively low critical solid-state thermal transition temperatures (e.g., melting temperature for semicrystalline material or glass transition temperature for amorphous material), at or below about 190° C.

First tubular layer 113, which will typically be considerably thinner than second tubular layer 130, may be pre-formed of a semicrystalline, relatively lubricious fluoropolymer (or fluoroplastic) such as polytetrafluoroethylene (PTFE) (e.g., via ram/paste extrusion); or fluorinated ethylene-propylene copolymer (FEP), perfluoroalkoxyethylene (PFA), poly(ethylene-co-tetrafluoroethylene) (ETEF), and the like (e.g., via melt tubing extrusion). These materials are desirable in light of their relatively high thermophysical stability (e.g., melting temperature well above 200° C.), particularly relative to the materials used to form second tubular layer 130.

In certain embodiments of the disclosure, a fluoroplastic first tubular layer 113 is chemically functionalized with surface treatment, such as chemical etching, plasma and corona treatment, and the like, to facilitate chemical bondability with second tubular layer 130 when forming deflectable shaft segment 100.

Thermoplastic polyesters such as poly(ethylene terephthalate) and polybutylene terephthalate, and polyamides such as nylon 6, nylon 66, and the like, can also be used to form first tubular layer 113, insofar as these thermoplastics also have relatively high, solid-state thermal transitions (that is, melting temperatures) well above 200° C.

In other aspects of the disclosure, first tubular layer 113 can be a preformed, heat-shrinkable tubing having an outer surface thermally bondable to second tubular layer 130, such that it is able to radially shrink down to tightly fit and geometrically conform to the circular perimeter of strut 110, thus forming an integrated strut assembly 111, when second tubular layer 130 is rendered molten in the manufacture of deflectable shaft segment 100 by means of melt reflow or thermal fusion bonding. Such a heat-shrinkable first tubular layer 113 exhibits enhanced thermophysical stability because of its lightly crosslinked polymer structure.

Consistent with the desirable material properties discussed above, strut 110 can be formed of an elastomeric polymer material, including thermoplastic elastomers or thermosetting elastomers (commonly known as rubber) that possesses a considerably higher thermophysical stability than the material(s) used in second tubular layer 130. In particular, suitable polymeric materials for forming strut 110 are thermosetting elastomers or rubbers selected from urethane/urea rubbers; silicone rubbers; isobutylene-based butyl rubbers; neoprene or polychloroprene rubbers; ethylene-propylene-diene rubber; nitrile rubber; acrylonitrile butadiene rubber; fluoroelastomers based on vinylidene fluoride, hexafluoropropylene, tetrafluoroethylene, perfluoromethylvinyleteher and the like. Strut 110 made of such a rubber material not only possesses an exceptionally high, solid-state thermophysical stability, but also exhibits outstanding hyperelasticity (e.g., a relatively low flexural modulus but a relatively high resiliency of material).

Unlike thermoplastic polymer resin materials, which largely lack chemical reactivity, a thermosetting polymer material is often commercially supplied as a reactive polymer system comprised of one or more chemically stable parts in raw state (e.g., liquid, paste, or gum-like states). A reactive mixture in preset ratios of individual parts is prepared for such a reactive polymer system, and then utilized to form strut 110 through rubber manufacturing methods that will be familiar those of the ordinary skill in the art, including reactive or rubber extrusion, reactive or rubber injection molding, reactive or rubber compression molding, mold casting, and the like. During rubber manufacturing, strut 110 formed or shaped of such a reactive rubber system is chemically converted into a solid, thermosetting elastomer or rubber material based on the underlying cure or vulcanization mechanism of the system.

In further embodiments of the disclosure, strut 110 may be formed of a thermoplastic elastomer material that exhibits superior thermophysical stability relative to the thermoplastic elastomer material(s) used in second tubular layer 130. Moreover, it is desirable for first tubular layer 113 to possess a superior, or at least equivalent, thermophysical stability (e.g., critical solid-state thermal transition temperature) relative to strut 110. For example, strut 110 may be formed of a polyester-based TPE material (e.g., Hytrel®, Arnitel®, etc.) with a relatively high thermophysical stability (e.g., melting temperature above about 200° C.), while second tubular layer 130 may be formed of one or more polyamide-based TPE materials (e.g., Pebax®, Vestamid® E, etc.), which have relatively lower thermophysical stability (e.g., melting temperatures well below about 200° C., such as within a range of about 134 to about 172° C.).

Polyester-based and polyamide-based TPE materials (e.g., Hytrel®, Arnitel®, and the like) have similar, flexible, amorphous polyether blocks (e.g., poly(tetramethylene oxide)) imparting elastomer-like mechanical properties of material, but differ in their respective, hard, semicrystalline blocks largely imparting the material's thermophysical stability (or critical solid-state thermal transition temperatures). Polyester-based TPE materials comprise semicrystalline polyester blocks whose melting temperature is about 223° C., while polyamide-based TPE materials comprise semicrystalline polyamide 12 (or nylon 12), whose melting temperature is about 178° C. Thus, at a relatively low melt/reflow temperature (e.g., a temperature near or slightly below about 200° C. but above about 178° C.) used when forming deflectable shaft segment 100, second tubular layer 130, which is preformed of various polyamide-based TPE materials, melts, redistributes, and is melt-bonded/coupled to strut assembly 111, while strut assembly 111 (including strut 110 and first tubular layer 113) generally retains its solid-state physical/geometric integrity in its entirety.

If different, but chemically compatible, TPE materials are used to form strut 110 and second tubular layer 130, along with first tubular layer 113 being formed of a thermoplastic material having equivalent or even superior thermophysical stability to strut 110 (e.g., polyester, or suitable polyester-based thermoplastic elastomer), as given in the above exemplary case, this can desirably facilitate the manufacturing of deflectable shaft segment 100 via a series of melt processing methods, such as melt extrusion, reflow, and/or thermal lamination, and the like, that will be familiar to those of the ordinary skill in the art.

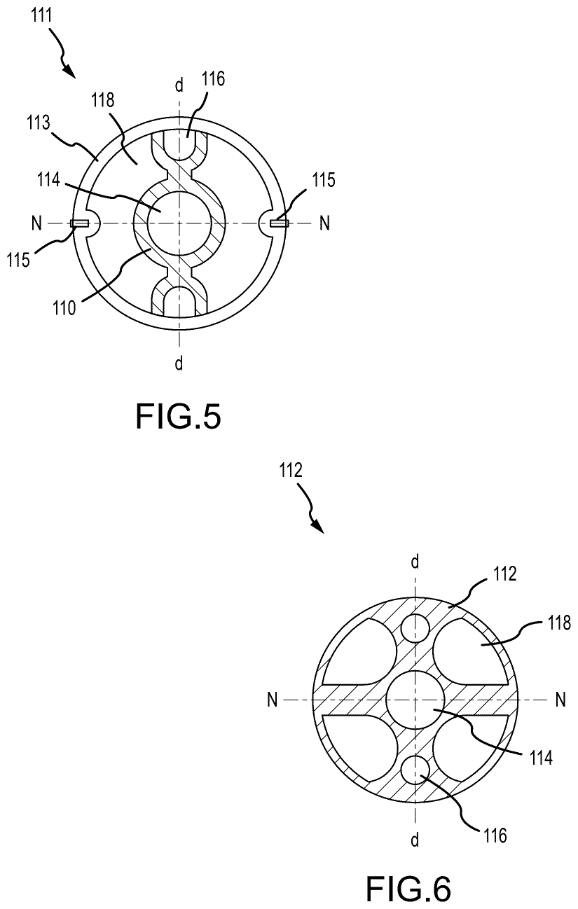

FIG. 6 is a cross-section of a strut assembly 112 within deflectable shaft segment 100 according to an alternative embodiment of the instant disclosure. In particular, FIG. 6 depicts an integral strut assembly 112 where the first tubular layer 113 and strut 110 are integrally and simultaneously formed of a single polymeric material.

Similar to strut 110, various elastomeric materials can be used to form an integral strut assembly 112 using a number of corresponding manufacturing approaches. For example, where a TPE material is chosen, an integral strut assembly 112 can be formed using a melt process, such as melt reflow, profile tubing extrusion, insert injection molding, or the like. Alternatively, where a thermosetting rubber is chosen, an integral strut assembly 112 can be formed using a reactive shaping process, such as liquid cast molding, reactive profile extrusion, reactive insert molding, or the like. Insofar as these manufacturing techniques will be familiar to those of ordinary skill in the art, further description thereof is not required herein.

Similar to strut assembly 111 (which includes separately-formed strut 110 and first tubular layer 113 as shown and described in connection with FIG. 4), an integral strut assembly 112 as depicted in FIG. 6 can be used to form a deflectable shaft segment 100 through a thermal lamination or reflow method as discussed above. That is, a second tubular layer 130 may be applied over an integral strut assembly 112, with an optional reinforcing braid layer 120 interposed therebetween, and the various components can then be integrated to form a deflectable shaft segment 100 via thermal fusion bonding, reflow, thermal lamination, or another suitable bonding technique, as will be familiar to those of the ordinary skill in the art.

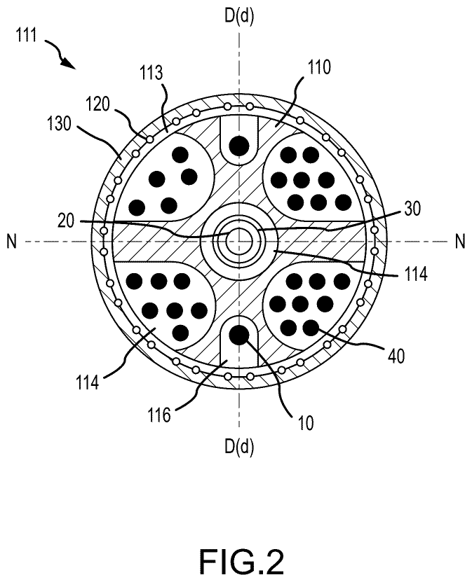

Various conductors, mechanically interactive elements (e.g., loop-activating wires, pull wires, and the like), and other catheter components can be installed into internal lumens 114, 116, 118 of catheter shaft 55 containing an integrated or integral strut assembly 111 or 112 via systematic stringing during catheter assembly. For example, as shown in FIG. 2, pull wires 10 can be routed through biased lumens 116, while loop-actuating wire 20 (optionally wholly or partially enclosed within compression coil 30) can be routed through central lumen 114.

Placement of loop-activating wire 20 within central lumen 14 along the centerline of catheter shaft 55 advantageously curtails the potential for any mechanical interference to a deflected shaft configuration by the actuation of loop-activating wire 20, and conversely, to a tightened/expanded loop of circular loop segment 230 by the pulling of a pull wire 10. Central lumen 114 can also accommodate other components, such as irrigation tubing in the case of an irrigated radiofrequency ablation catheter.

Electrical conductors 40 can be routed through side lumens 118. Isolating electrical conductors 40 inside lumens 118 advantageously minimizes the potential for damage to electrical conductors 40 as a result of mechanical manipulations of pull wires 10 and/or loop-actuating wire 20 during a procedure (e.g., as necessary to navigate catheter 5 through the vasculature and into the heart).

Although several embodiments have been described above with a certain degree of particularity, those skilled in the art could make numerous alterations to the disclosed embodiments without departing from the spirit or scope of this invention.

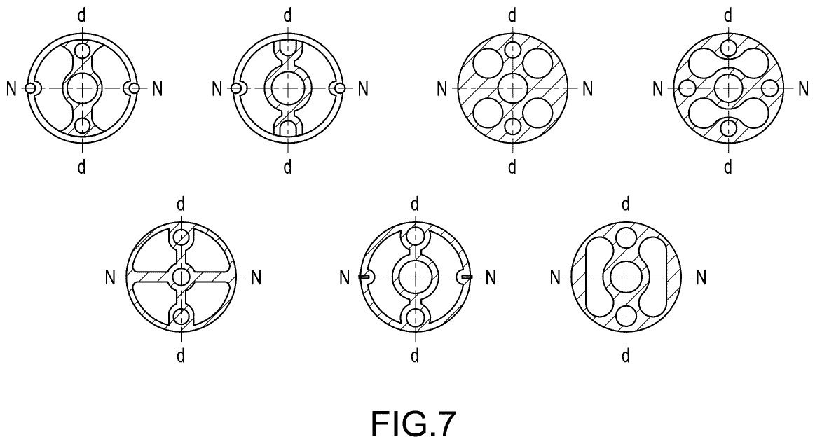

For example, FIGS. 2, 4, 5, and 6 illustrate various cross-sectional configurations of strut 110, but it should be understood that these configurations are merely exemplary and that additional configurations are within the spirit and scope of the present disclosure. In this regard, additional exemplary cross-sectional configurations are shown in FIG. 7. Of course, it should be understood that any of these exemplary cross-sectional configurations could be formed either from a strut 110 and a separate first tubular 113 or as an integrally formed strut assembly 112. Similarly, any of the exemplary cross-sectional configurations could incorporate one or more walls 117 and/or one or more biasing structures 115 to promote planar deflection about neutral axis N-N within deflection plane D-D or along deflection axis d-d. In short, the various features and aspects disclosed herein can be combined in any fashion in order to create any number of catheter shaft configurations.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A catheter shaft, comprising:
a single piece strut having a central portion defining a central lumen extending therethrough and a pair of opposing arms extending radially away from the central portion, each of the pair of opposing arms defining a respective biased lumen extending therethrough; and
a first tubular layer surrounding the strut and defining two opposing side lumens extending through the catheter shaft between the first tubular layer and the strut.

2. The catheter shaft according to claim 1, wherein the first tubular layer is integrally formed with the strut from a single polymeric material.

3. The catheter shaft according to claim 1, wherein the strut comprises a first polymeric material, the first tubular layer comprises a second polymeric material different from the first polymeric material, and the strut is bonded to the first tubular layer.

4. The catheter shaft according to claim 1, further comprising a second tubular layer surrounding the first tubular layer.

5. The catheter shaft according to claim 4, wherein a material of which the second tubular layer is formed has a lower critical solid-state thermal transition temperature than a material of which the strut is formed.

6. The catheter shaft according to claim 4, further comprising a reinforcing layer disposed between the first tubular layer and the second tubular layer.

7. The catheter shaft according to claim 6, wherein the reinforcing layer comprises a braided metallic mesh.

8. The catheter shaft according to claim 1, wherein a line extending through the biased lumens and the central lumen defines a deflection axis, a line extending through a center of the central lumen and perpendicular to the deflection axis defines a neutral axis, and an area moment of inertia of the strut about the neutral axis is less than or equal to an area moment of inertia of the strut about the deflection axis.

9. The catheter shaft according to claim 8, further comprising a pair of biasing ribbons positioned along the neutral axis equidistant from the deflection axis.

10. The catheter shaft according to claim 9, wherein the pair of biasing ribbons comprises a pair of metallic ribbons.

11. The catheter shaft according to claim 1, wherein the strut further comprises a pair of walls extending radially away from the central portion to the first tubular layer and dividing each of the two opposing side lumens into a respective pair of sub-lumens.

12. The catheter shaft according to claim 1, further comprising:
a first pull wire extending through a first biased lumen;
a second pull wire extending through a second biased lumen; and
a loop-activating wire extending through the central lumen.

13. The catheter shaft according to claim 12, further comprising a plurality of electrical conductors extending through at least one of the two opposing side lumens.

14. A method of manufacturing a catheter shaft, comprising:
forming a single-piece strut having a central portion defining a central lumen extending therethrough and a pair of opposing arms extending radially away from the central portion, each of the pair of opposing arms defining a respective biased lumen extending therethrough; and
forming a first tubular layer around the strut to define two opposing side lumens extending through the catheter shaft between the first tubular layer and the strut.

15. The method according to claim 14, wherein forming the single-piece strut and forming the first tubular layer around the strut comprise integrally forming the single-piece strut and the first tubular layer from a single polymeric material.

16. The method according to claim 14, wherein:
forming the single-piece strut comprises forming the single piece strut from a first polymeric material;
forming the first tubular layer comprises forming the first tubular layer from a second polymeric material different from the first polymeric material; and
wherein the method further comprises bonding the first tubular layer to the single-piece strut.

17. The method according to claim 14, further comprising forming a second tubular layer around the first tubular layer.

18. The method according to claim 17, wherein a material of which the second tubular layer is formed has a lower critical solid-state thermal transition temperature than a material of which the first tubular layer is formed.

19. The method according to claim 17, further comprising forming a reinforcing layer between the first tubular layer and the second tubular layer.

20. The method according to claim 14, wherein the single-piece strut defines a deflection axis extending through the biased lumens and the central lumen and a neutral axis perpendicular to the deflection axis, and wherein an area moment of inertia of the strut about the neutral axis is less than or equal to an area moment of inertia of the strut about the deflection axis.

21. A catheter shaft formed according to a process comprising:
forming a single-piece strut having a central portion defining a central lumen extending therethrough and a pair of opposing arms extending radially away from the central portion, each of the pair of opposing arms defining a respective biased lumen extending therethrough;
forming a first tubular layer around the strut to define two opposing side lumens extending through the catheter shaft between the first tubular layer and the strut;
disposing a first pull wire in a first biased lumen;
disposing a second pull wire in a second biased lumen; and
disposing a loop-activating wire in the central lumen.

22. The method according to claim 21, wherein the single-piece strut defines a deflection axis extending through the biased lumens and the central lumen and a neutral axis perpendicular to the deflection axis, and wherein an area moment of inertia of the strut about the neutral axis is less than or equal to an area moment of inertia of the strut about the deflection axis.
